(19) United States
(12) Patent Application Publication
(10) Pub. No.: US 2026/0014016 A1
(43) Pub. Date: Jan. 15, 2026

(54) DEVICE FOR LENGTHENING AND/OR STRAIGHTENING A PENIS

(71) Applicant: MSP Concept GmbH & Co. KG, Berlin (DE)

(72) Inventor: Matthias SUCHY, Berlin (DE)

(73) Assignee: MSP Concept GmbH & Co. KG, Berlin (DE)

(21) Appl. No.: 19/264,117

(22) Filed: Jul. 9, 2025

(30) Foreign Application Priority Data
Jul. 10, 2024 (DE) 102024119638.6

(51) Int. Cl. A61F 5/41

(52) U.S. Cl. CPC A61F 5/41; A61F 2005/411

(57) ABSTRACT

A device for extending and/or straightening a penis includes a fastening element that fastens a penis, a traction device that is connected to the fastening element and that exerts a tractive force on a part of the penis, a tractive force measuring device that measures the tractive force exerted on the part of the penis in operation, an electronic component that ascertains a class assigned to the measured tractive force from plural classes including a class which classifies a tractive force assigned thereto as appropriate for operation, an output device that is connected to the fastening element and/or the traction device and that outputs the class haptically, acoustically, and/or visually when the measured tractive force is classified as appropriate for operation, and a power source for operating the electronic component, the tractive force measuring device, and the output device.

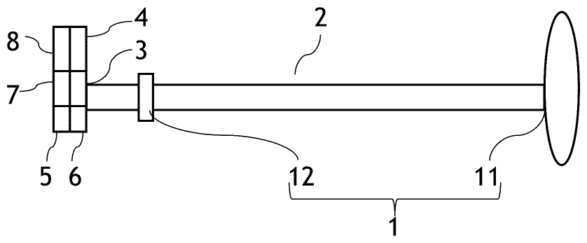

The invention relates to a device for extending and/or straightening a penis. In particular, the invention relates to a device for permanently extending and/or straightening a penis, with the aid of which it is possible without operative intervention, on the basis of a continuous extension of the penis by action of a stretching force, to achieve a lasting extension and/or lasting straightening. Such devices are also referred to as penis extension devices.

Such devices are known from EP 1 480 586 A1 and DE 10 2020 105 224 A1. The devices contain a fastening element, which is designed to fasten a penis, a traction device in the form of stretching rods, which is connected to the fastening element and is designed to exert a tractive force on at least a part of the penis. The fastening element and the traction device together form a penis extension and/or straightening device, which causes the lengthening and/or straightening of the penis.

Furthermore, a fastening device is known from EP 3 463 218 B1, which contains a coupling element and a tractive force measuring device and which is suitable for transmitting a tractive force of a penis extension device to a penis, wherein an electronic component and a power source are present. The tractive force measuring device is designed to electrically or electronically ascertain a measured value for the exerted traction together with the electronic component. The electronic component comprises a radio device for transmitting the ascertained measured value to an external receiver, wherein the radio device is designed as a Bluetooth device.

However, there is still a need to provide a user-friendly penis extension and/or straightening device, which enables pain-free and effective use and is easy to handle.

It is an object of the invention to provide a device for extending and/or straightening a penis, which is pain-free and is suitable for the permanent extension and/or straightening of a penis in a manner harmless to physical integrity. At the same time, its handling is to be easy and convenient.

The object is achieved according to the invention by a device having the features of claim 1. Advantageous refinements and modifications are specified in the dependent claims.

The invention relates to a device for extending and/or straightening a penis, containing
a fastening element, which is designed to fasten a penis,
a traction device, which is connected to the fastening element and is designed to exert a tractive force or tensile stress on at least a part of the penis,
a tractive force measuring device, which is designed to measure the tractive force exerted on the at least one part of the penis during operation,
an electronic component, which is designed to ascertain a class assigned to the measured tractive force from a plurality of classes, wherein the multiple classes comprise a class which classifies a tractive force assigned thereto as appropriate for operation,
an output device, which is connected to the fastening element and/or the traction device and is designed to haptically, acoustically, and/or visually output the class ascertained by the electronic component when it classifies the measured tractive force as appropriate for operation,
a power source for operating the electronic component, the tractive force measuring device, and the output device.

The output device is in particular mechanically connected to the tractive force measuring device and is therefore in particular a part of the device. It is therefore made possible for a user of the device to verify that the exerted tractive force is pain-free and is suitable for the permanent extension and/or straightening of a penis in a manner harmless to physical integrity. The user can thus be certain that he will also not have any physical damage and/or complaints due to continuous use. It is typical for the user of such a device to wear it multiple hours a day if possible. It is therefore essential for the user to know whether the exerted tractive force is also suitable for a longer duration. Furthermore, it is also helpful for the user to ensure that the set tractive force is sufficient to achieve the effect desired by him.

In one preferred embodiment, the output device is furthermore designed to acoustically and/or visually output the class ascertained by the electronic component if it classifies the measured tractive force as inappropriate for operation. The output of the ascertained class which classifies the measured tractive force as inappropriate for operation differs acoustically and/or visually from the output of the class which classifies the measured tractive force as appropriate for operation. The user is thus actively notified of whether the exerted tractive force is unsuitable or suitable for continuous use of the device. The acoustic and/or visual distinction can be implemented by the output of different colors, symbols, tones, tone sequences, and the like.

Alternatively or additionally to the acoustic and/or visual output, the output device can be designed to output a haptic signal. Haptic output devices comprise any devices which give tactile feedback which is perceived by the sense of touch. This includes vibrations, forces exerted on the skin, or electrical pulses generated by stimulation current. Such devices can be used in various applications, among them mobile devices, game consoles, medical devices, and virtual reality systems, to supply haptic feedback.

The output device is preferably designed as an optical display device, which is designed to output the ascertained class by means of an optical code having color and/or symbol information. Only the class which is assigned to a tractive force classified as appropriate for operation can be assigned the optical code. An individualizing optical code is preferably assigned to each ascertainable class. The classes are easily distinguishable by such an optical code. The optical code can be produced in particular in the form of numeric or alphanumeric characters, for example by means of a display.

As already mentioned, the electronic component is designed to ascertain a class for the measured tractive force which is assigned to this tractive force. The assignment or the ascertainment can be carried out, for example, by means of hardwired structural elements. If the tractive force is represented in the electronic component by a measured voltage or amperage, for example, an association with the respective class or the respective classes can then take place by means of threshold value comparison. This can be implemented both in an analog and in a digital circuit.

According to one preferred embodiment, a memory can be provided and the electronic component can be designed to progressively store measured tractive force values, in particular tractive force values measured at regular intervals, in the memory. In this way, the device can create a record of tractive forces that is readable or retrievable thereafter. Alternatively or additionally, the memory can record how long the tractive force was located within which tractive force range, for example on the basis of the assignment to the respective classes.

A memory is preferably provided, in which tractive forces are stored. By means of the memory, each tractive force is assigned in each case one class of the plurality of classes, wherein one of the classes classifies at least one of the stored tractive forces, to which it is assigned, as appropriate for operation. In this case, the electronic component is preferably designed to compare the measured tractive force with the tractive forces stored in the memory and in this way to ascertain the class assigned thereto. Specific tractive force ranges are preferably each assigned to one class.

The stored tractive forces are preferably classified into tractive forces appropriate for operation and into tractive forces inappropriate for operation, the latter in particular into tractive forces excessively high for operation and into tractive forces excessively low for operation. Preferably, three classes are stored in the device in particular in the memory, namely one class which is assigned to tractive forces appropriate for operation, another class which is assigned to tractive forces excessively high for operation, and still another class which is assigned to tractive forces excessively low for operation.

It can therefore be indicated effectively and easily to the user whether he achieves a suitable effect by means of the tractive forces appropriate for operation, a damaging effect by means of the tractive forces excessively high for operation, or an unsatisfactory effect or no effect by means of the tractive forces excessively low for operation by means of use of the device. In penis extension devices, the set tractive force is typically specified in g (grams). For example, a range between 600 g to 800 g can be considered to be a suitable tractive force range, while a tractive force of less than 500 g or 600 g is considered to be excessively low and a tractive force of greater than 800 g or 900 g is considered to be excessively high.

The output device is preferably designed as an optical display device for displaying color information. The display device is preferably designed as electronic display means for example in the form of a display and/or as illuminant, in particular as an LED (light-emitting diode) illuminant. In one preferred embodiment, the output device is designed as a bar display and/or as a multicolored light-emitting diode. It can thus display multiple classes. The display device is preferably designed as an illuminant actuatable in multiple colors, in particular as light-emitting diode devices. The display device is preferably designed as an RGB LED device, which can display a large number of different colors and/or color profiles depending on the actuation. The display device is thus designed to be cost-effective and functionally flexible with a low power consumption at the same time.

The device preferably furthermore has an input element, in particular an input button, which is designed to activate the output device and/or to permit the assignment of a class to the stored tractive forces to be set. The device is designed as power saving in that the user controls the activation of the output device. Alternatively or additionally, the user can set the assignment of a class to the stored tractive forces. The user can thus store and/or change the assignment of the respective class to one of the stored tractive forces depending on the constitution of his penis. The input element is preferably designed to permit the class assigned to the respectively stored tractive force to be changed. That is to say, the classes are stored, but can be changed as needed by the user.

In one preferred embodiment, the input element is designed to activate the output device or to permit the assignment of the class to the stored tractive forces to be set depending on a duration in which the input element is actuated. Both functions are thus implemented in a simple manner. For example, the input element is designed to activate the output device when it is actuated over a predetermined duration and to permit the assignment of the class to the stored tractive forces to be set if it is pressed over a further predetermined duration which is different from the predetermined duration. For example, the predetermined duration is 1 second while the further predetermined duration is 3 seconds.

In the simplest form, the input element can be a button or a switch. The input element can also be a keyboard. Other suitable input elements are also advantageous such as touch sensors, proximity sensors, or touchpads. In particular, the output device and the input element can be combined to form a touch display.

In one preferred embodiment, the tractive force measuring device, the memory, the electronic component, the power source, and the output device are permanently connected to the fastening element and/or the traction device. They are preferably permanently connected to the traction device. All mentioned parts of the device thus form a fixed unit. It is thus ensured that the tractive force measuring device, the memory, the electronic component, the power source, and the output device are permanently integrated in the penis extension and/or straightening device.

In one preferred embodiment, the memory is furthermore designed to store an operating duration and/or an application duration of the device of the electronic component, and/or the output device is designed to output the operating duration and/or the application duration of the device. His user behavior, in particular his usage duration, can thus be communicated to the user.

The electronic component is preferably designed to regulate or control the output device such that it exclusively outputs the operating duration and/or the application duration of the device after input of a predetermined code. It is thus ensured that only an authorized person can view these data.

The output device is designed to output the usage duration visually and/or acoustically. The output device is preferably designed to output the operating duration and/or the application duration of the device in the form of an optical code, which has a flashing pattern. Flashing patterns are easily distinguishable and memorable for a user. The flashing pattern is preferably generated by activating single-colored or multicolored LEDs of the output device at different times. For example, the output device is designed to indicate a predetermined time interval with each flash. For example, each flash indicates a usage duration of one hour, so that flashing eight times indicates a usage duration of eight hours a day. Alternatively, the output device can also be designed to output the usage duration acoustically via speech information or a tone code, for example via a sequence of successive tones.

In the present sense, the operating duration describes the period of time over which the device is switched on, while the application duration preferably describes the period of time during which a tractive force acts on the penis, preferably a tractive force classified as appropriate by the electronic component. Preferably, one or more of the following items of information is stored in the memory and/or the output device is designed to output one or more of the following items of information: the operating duration, the application duration, the period of time during which the measured tractive force is ascertained as assigned to a first, second, and/or a third class, wherein the first class corresponds to an appropriate classification, while the second and the third class respectively correspond in an assigned manner to an excessively low tractive force and an excessively high tractive force.

It can be ascertained for example by means of a threshold value comparison that the device is in application and is not solely in operation. In this case, for example, the device can be considered to be in application if the tractive force exceeds a specific minimum value, for example 50 g.

The fastening element and the device are preferably designed such that they are adaptable to different penis lengths. Together they form a penis extension and/or straightening device, i.e. a unit which provides a penis extension and/or straightening function. Penis extension and/or straightening devices can be implemented in various ways and are known from the prior art. During operation, the user himself sets the tractive force which is exerted on at least one part of the penis by means of the tractive force device.

The penis has various sections. In particular, it has a proximal section, an intermediate section, and a distal section. The proximal section is a section of the penis, which is arranged closest to the body, in particular originating from the body, and preferably has the base of the penis. The intermediate section is preferably a section which lies between the proximal section and the distal section, such as the penis shaft. The distal section is a section of the penis which is arranged at a distance from the body in relation to the proximal section and the intermediate section. It comprises, for example, the glans penis and/or an area adjoining thereto.

The fastening element is designed to fasten the penis of the user. The fastening element is preferably formed in multiple parts. It preferably has a support element, preferably a support ring, to be arranged on a proximal section of the penis and a fixing means, which is designed to fix a distal section of the penis. A secure fastening of the penis on the device is thus ensured. The support element is used to support the device on the body of the user.

The traction device, which is connected to the fastening element and is designed to exert a tractive force on at least a part of the penis, is preferably designed in the form of stretching rods extending in parallel. The stretching rods preferably extend parallel to the intermediate section, away from the support element. The stretching rods can be designed as articulated or rigid. Furthermore, they can each be formed in one part or multiple parts.

The tractive force measuring device, which is designed to measure the tractive force exerted on the at least one part of the penis during operation, can be implemented in various ways. Tractive force measuring devices are generally known.

The memory, in which tractive forces are stored, to each of which one class of a plurality of classes is assigned, is preferably designed as an internal memory of the device. That is to say, it is not transportable, but rather is designed as a fixed memory of the device. The memory can be designed as part of the electronic component or a component separate therefrom.

The electronic component, which is designed to compare the measured tractive force with the tractive forces stored in the memory and to ascertain the class assigned thereto, can be designed, for example, as a microcontroller. The electronic component contains a regulating and/or control unit, which is designed to regulate or to control the tractive force measurement, the comparison of the measured tractive force with the tractive forces stored in the memory, and the ascertainment of the assigned class and the output of the ascertained class by means of the output device.

The power source, which is designed to operate the electronic component and optionally the memory, is a battery or preferably a rechargeable energy storage device.

The invention is explained in more detail hereinafter with reference to an appended drawing. In the schematic figure, which is not to scale

The Figure shows a side view of a device according to the invention.

The Figure shows a side view of a device according to the invention. The device is intended for extending and/or straightening a penis and contains a fastening element 1, which is designed to fasten a penis. The fastening element 1 has a support element 11 in the form of a support ring to be arranged on a proximal section (not shown) of a penis (not shown) and a fixing means 12, which is designed to fix a distal section (not shown) of the penis. Furthermore, the device has a traction device 2 in the form of stretching rods extending in parallel. The stretching rods can be in one part or multiple parts and can extend linearly or can be articulated, they are shown extending linearly here by way of example. The traction device 2 is connected to the fastening element 1 and is designed to exert a tractive force on at least a part of the penis. The fastening element 1 and the traction device 2 are used as the penis extension and/or straightening device.

The device furthermore has a tractive force measuring device 3, which is designed to measure the tractive force exerted on the at least one part of the penis during operation, and a memory 4, in which tractive forces are stored, to each of which one class of a plurality of classes is assigned. One of the classes classifies at least one of the stored tractive forces, to which it is assigned, as appropriate for operation. Furthermore, the device has an electronic component 5, which is designed to compare the measured tractive force with the tractive forces stored in the memory and to ascertain the class assigned thereto, a power source 6 for operating the electronic component 5, and an output device 7, which is designed to output the class ascertained by the electronic component acoustically and/or visually if the tractive force is classified as appropriate for operation. The electronic component 5 has a regulating and/or control device (not shown), which is designed to regulate or control the tractive force measurement, the comparison of the measured tractive force with the tractive forces stored in the memory 4, and the ascertainment of the assigned class and the output of the ascertained class by means of the output device 7.

The tractive force measuring device 3, the memory 4, the electronic component 5, the power source 6, and the output device 7 form a device which are permanently connected to the traction device 2.

### LIST OF REFERENCE SIGNS

1 fastening element
11 support element
12 fixing element
2 traction device
3 traction measuring device
4 memory
5 electronic component
6 power source
7 output device
8 input element

1. A device for extending and/or straightening a penis, the device comprising:
a fastening element, which is configured to fasten a penis,
a traction device, which is connected to the fastening element and is configured to exert a tractive force on at least one part of the penis,
a tractive force measuring device, which is configured to measure the tractive force exerted on the at least one part of the penis in operation,
an electronic component, which is configured to ascertain a class assigned to the measured tractive force from a plurality of classes, the plurality of classes comprising a class which classifies a tractive force assigned thereto as appropriate for operation,
an output device, which is connected to the fastening element and/or the traction device and is configured to output the class ascertained by the electronic component haptically, acoustically, and/or visually when the measured tractive force is classified as appropriate for operation, and
a power source that supplies power to the electronic component, the tractive force measuring device, and the output device.

2. The device as claimed in claim 1, further comprising a memory, in which tractive forces are stored, to each of which one class of the plurality of classes is assigned,
wherein one of the classes classifies at least one of the stored tractive forces, to which the one of the classes is assigned, as appropriate for operation, and
wherein the electronic component is configured to compare the measured tractive force with the tractive forces stored in the memory and to ascertain the class assigned thereto.

3. The device as claimed in claim 1, wherein the output device is furthermore configured to output the class ascertained by the electronic component haptically, acoustically, and/or visually when the measured tractive force is classified as inappropriate for operation, and
wherein the output of the ascertained class which classifies the measured tractive force as inappropriate for operation differs haptically, acoustically, and/or visually from the output of the class which classifies the measured tractive force as appropriate for operation.

4. The device as claimed in claim 1, wherein the output device comprises an optical display device, which is configured to output the ascertained class by an optical code which has color and/or symbol information.

5. The device as claimed in claim 4, wherein the output device comprises a bar display and/or a multicolored light-emitting diode.

6. The device as claimed in claim 2, further comprising an input element, which is configured to activate the output device and/or to permit an assignment of a class to the stored tractive forces to be set.

7. The device as claimed in claim 6, wherein the input element is configured to activate the output device and/or to permit the assignment of the class to the stored tractive forces to be set depending on a duration in which the input device is actuated.

8. The device as claimed in claim 2, wherein the memory is furthermore configured to store an operating duration of the electronic component and/or an application duration of the device, and
wherein the output device is configured to output the operating duration.

9. The device as claimed in claim 8, wherein the electronic component is configured to regulate or control the output device such that the output device outputs the operating duration and/or an application duration of the device exclusively after input of a predetermined code.

10. The device as claimed in claim 8, wherein the output device is configured to output the operating duration and/or an application duration of the device in the form of an optical code, which has a flashing pattern.

11. The device as claimed in claim 6, wherein the input element is an input button.
